(19) United States
(12) Patent Application Publication
(10) Pub. No.: US 2023/0263513 A1
(43) Pub. Date: Aug. 24, 2023

(54) SYSTEM, CANNULA, AND METHOD FOR DE-AIRING A PATIENT'S HEART

(71) Applicants: THE CLEVELAND CLINIC FOUNDATION, Cleveland, OH (US); PARKER HANNIFIN CORPORATION, Cleveland, OH (US)

(72) Inventors: Jose L. Navia, Cleveland, OH (US); Ricardo Gonzalez, Valparaiso, IN (US); Ferguson J. Poliquin, Cleveland, OH (US)

(21) Appl. No.: 18/111,090

(22) Filed: Feb. 17, 2023

Related U.S. Application Data
(60) Provisional application No. 63/311,534, filed on Feb. 18, 2022.

(51) Int. Cl. A61B 17/00

(52) U.S. Cl. CPC A61B 17/00234; A61B 2017/00247

(57) ABSTRACT

A cannula, system, and method for de-airing a patient's heart following surgery are described. The cannula includes a tubular wall having a distal end portion and a proximal end portion. The wall also has a length extending from the distal end portion to the proximal end portion. The wall at least partially defines a first lumen extending lengthwise of the wall into the distal end portion and terminating in a closed first end. The wall also at least partially defines a second lumen extending lengthwise of the wall and terminating in a closed second end. The wall is configured and dimensioned such that the cannula can be inserted into the patient's heart. A first aperture extends through the wall from the first lumen to an exterior surface of the wall. A second aperture extends through the wall from the second lumen to the exterior surface of the wall.

### RELATED APPLICATION

This application claims priority from U.S. Provisional Application No. 63/311,534, filed 18 Feb. 2022, the subject matter of which is incorporated herein by reference in its entirety.

### FIELD OF THE INVENTION

The present invention relates to a system, cannula, and method for de-airing a patient's heart following surgery and, more particularly, to a system and method in which a plural lumen cannula is inserted into the patient's heart to conduct fluid, such as gas, to the patient's heart and to apply suction to the patient's heart.

### BACKGROUND OF THE INVENTION

Air embolisms are a known complication of cardiovascular surgery and may produce injury to a patient. Air embolisms can result from air being trapped in a patient's heart as a consequence of surgery. Various procedures and systems have previously been proposed to prevent or minimize air from being trapped in a patient's heart and/or to remove or dislodge air that becomes trapped in a patient's heart.

### SUMMARY

In an aspect, alone or in combination with any other aspect, a cannula for de-airing a patient's heart following surgery is described. The cannula includes a tubular wall having a distal end portion and a proximal end portion. The wall also has a length extending from the distal end portion to the proximal end portion. The wall at least partially defines a first lumen extending lengthwise of the wall into the distal end portion and terminating in a closed first end. The wall also at least partially defines a second lumen extending lengthwise of the wall and terminating in a closed second end. The wall is configured and dimensioned such that the cannula can be inserted into the patient's heart. A first aperture extends through the wall from the first lumen to an exterior surface of the wall. A second aperture extends through the wall from the second lumen to the exterior surface of the wall.

In an aspect, alone or in combination with any other aspect, a system for de-airing a patient's heart following surgery is described. The system includes a source of fluid, a source of suction, and a cannula configured and dimensioned to be selectively inserted into the patient's heart and to selectively conduct fluid from the source of fluid to the patient's heart. The cannula has a distal end portion and a proximal end portion. The cannula also has a length extending from the distal end portion to the proximal end portion. The cannula includes a wall extending lengthwise of the cannula. The wall at least partially defines a first lumen extending lengthwise of the cannula into the distal end portion and terminating in a closed first end. The wall also at least partially defines a second lumen extending lengthwise of the cannula and terminating in a closed second end located proximally of the closed first end of the first lumen. The cannula also includes a first aperture extending through the wall of the cannula from the first lumen to an exterior surface of the wall and a second aperture extending through the wall of the cannula from the second lumen to the exterior surface of the wall. A control apparatus is operable to control pressure and flow of fluid from the source of fluid to the cannula and to control application of suction from the source of suction to the cannula. The control apparatus is in fluid communication with the source of fluid and with the cannula. The control apparatus also is in fluid communication with the source of suction and with the cannula. The control apparatus is operable to direct fluid from the source of fluid to the first lumen and/or the second lumen of the cannula. The control apparatus also is operable to apply suction from the source of suction to the first lumen and/or the second lumen.

In an aspect, alone or in combination with any other aspect, a method of de-airing a patient's heart following surgery is described. A patient is placed in a Trendelenburg position with the patient's head disposed below the patient's feet. A plural lumen cannula is inserted into a left atrium of the patient's heart. The plural lumen cannula comprises a tubular wall at least partially defining a first lumen and at least partially defining a second lumen. A first aperture is formed in the wall adjacent a distal end of the plural lumen cannula and communicates with the first lumen. A second aperture is formed in the wall proximal of the first aperture and communicates with the second lumen. The plural lumen cannula is extended into a left ventricle of the patient's heart such that the first aperture is disposed in the left ventricle and the second aperture is located in the left atrium. Fluid is introduced into the left atrium through the second lumen and the second aperture. Fluid is suctioned from the left ventricle through the first aperture and the first lumen.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 is a schematic view of a system for de-airing a patient's heart following surgery in accordance with an embodiment of the present invention;

FIG. 2 is a schematic sectional view taken along line 2-2 of FIG. 1;

FIG. 3 is a schematic sectional illustration of a cannula of the system of FIG. 1 in the process of being inserted into a patient's heart;

FIG. 4 is a schematic exterior view of a patient's heart having the cannula of the system of FIG. 1 inserted in the patient's heart;

FIG. 5 is a schematic sectional illustration of a cannula of the system of FIG. 1 fully inserted into a patient's heart;

FIG. 6 describes an example method of use of the system of FIG. 1;

FIG. 7A is a schematic cross-sectional view of a cannula of a system for de-airing a patient's heart in accordance with an embodiment of the present invention, taken along line 7A-7A in FIG. 7D;

FIG. 7B is a schematic partial side view of the cannula of FIG. 7A;

FIG. 7C is a schematic partial side view of the cannula of FIG. 7A;

FIG. 7D is a schematic partial side view of the cannula of FIG. 7A;

FIG. 8 is a schematic side view of the cannula of FIG. 7A; and

FIG. 9 is a schematic diagrammatic view of example operational function of the cannula of FIG. 7A.

### DETAILED DESCRIPTION

Unless defined otherwise, all technical and scientific terms used herein have the same meaning as is commonly understood by one of ordinary skill in the art to which the present disclosure pertains.

As used herein, the singular forms “a”, “an”, and “the” can include the plural forms as well, unless the context clearly indicates otherwise. It will be further understood that the terms “comprises” and/or “comprising”, as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

It will be understood that when an element is referred to as being “on”, “attached” to, “connected” to, “coupled” with, “contacting”, “adjacent”, etc., another element, it can be directly on, attached to, connected to, coupled with, contacting, or adjacent the other element, or intervening elements may also be present. In contrast, when an element is referred to as being, for example, “directly on”, “directly attached” to, “directly connected” to, “directly coupled” with, “directly contacting”, or “directly adjacent” another element, there are no intervening elements present. It will also be appreciated by those of ordinary skill in the art that references to a structure or feature that is disposed “directly adjacent” another feature may have portions that overlap or underlie the adjacent feature, whereas a structure or feature that is disposed “adjacent” another feature might not have portions that overlap or underlie the adjacent feature.

Spatially relative terms, such as “under”, “below”, “lower”, “over”, “upper”, “proximal”, “distal”, and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that, although the terms “first”, “second”, etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

The invention comprises, consists of, or consists essentially of the following features, in any combination.

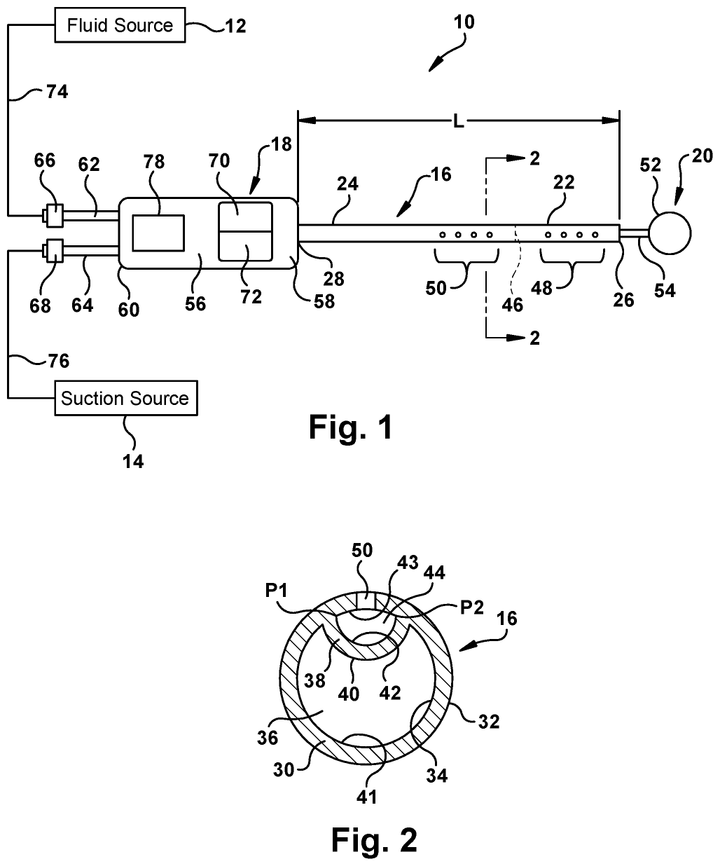

FIGS. 1 and 2 illustrate a system 10 for de-airing a patient's heart following surgery, in accordance with an example of the present invention. The system 10 comprises a source of fluid 12 (which may be, for example, at least one source of carbon dioxide), a source of suction 14 (which may be, for example, at least one vacuum line), a plural lumen cannula 16, and a control apparatus 18. The system may also comprise an atraumatic member 20, such as a balloon, mounted adjacent an end of the cannula 16.

The cannula 16 has a distal end portion 22 and a proximal end portion 24, which is spaced apart from the distal end portion. The cannula 16 also has a length L, which extends from the distal end portion 22 to the proximal end portion 24. More particularly, the length L of the cannula 16 extends from the distal end 26 of the cannula to the proximal end 28 of the cannula.

As best seen in FIG. 2, the cannula 16 includes a tubular outer wall 30, which extends along the length L of the cannula. The outer wall 30 is made of a flexible biocompatible material, for example, a biocompatible plastic, such as silicone. As used in this application, “flexible” means that a structure or material, such as the outer wall 30 or the material of which the outer wall is made, is capable of being flexed, which is to say capable of being turned, bowed, or twisted without breaking. The outer wall 30 has an exterior surface 32, which circumscribes the outer wall, and an interior surface 34, which extends parallel to the exterior surface. The exterior surface 32 and the interior surface 34, when viewed in cross-section taken perpendicular to the length L of the cannula 16, may be circular in shape, as shown in FIG. 2, but may have other closed configurations or shapes.

The interior surface 34 of the outer wall 30 of the cannula 16 partially defines a first lumen 36 of the cannula. The first lumen 36 extends along the length L of the cannula 16 and the length of the outer wall 30 from the proximal end portion 24 into the distal end portion 22. The first lumen 36 also extends along the length of the cannula 16 from the proximal end 28 to the distal end 26. The distal end of the first lumen 36 is closed. The distal end of the first lumen 36 may, for example, be closed by a wall (not shown), which extends transversely of the first lumen and the outer wall 30 at the distal end 26 of the cannula 16. The distal end of the first lumen 36 may alternatively, for example, be closed by a wall of the atraumatic member 20 mounted adjacent the distal end 26 and the distal end portion 22 of the cannula 16.

The cannula 16 also includes an inner wall 38, which extends lengthwise of the cannula. The inner wall 38, like the outer wall 30, is made of a flexible biocompatible material, for example, a biocompatible plastic, such as silicone, and may be molded in one piece with the outer wall. As can be seen in FIG. 2, the inner wall 38, when viewed in cross-section taken perpendicular to the length L of the cannula 16, extends in an arc from a first point P1 on the interior surface 34 of the outer wall 30 to a second point P2 on the interior surface of the outer wall. The second point P2 is spaced apart from the first point P1 around or along the interior circumference of the outer wall.

A first surface 40 of the inner wall 38 is presented toward a first portion 41 of the interior surface 34 of the outer wall 30 and, together with the first portion of the interior surface of the outer wall, defines the first lumen 36. An opposite second surface 42 of the inner wall 38 is presented toward a second portion 43 of the interior surface 34 of the outer wall 30 and, together with the second portion of the interior surface of the outer wall, defines a second lumen 44. The second lumen 44 extends lengthwise of the cannula 16 and the outer wall 30 from the proximal end portion 24 toward the distal end portion 22. The second lumen 44 also extends lengthwise of the cannula 16 from the proximal end 28 toward the distal end 26. The distal end of the second lumen 44 is closed. The distal end of the second lumen 44 is closed by a wall 46, which extends transversely of the second lumen and the length L of the cannula 16 and the outer wall 30 at a location proximal of the distal end portion 22 of the cannula. The second lumen 44 thus does not extend for the full length L of the cannula and the outer wall 30. The wall 46 may be molded in one piece with the inner wall 38 and with the outer wall 30.

In the distal end portion 22 of the cannula 16, a series of first ports or first apertures 48 extend through the outer wall 30 from the exterior surface 32 to the interior surface 34. Each of the first apertures 48 thus extends from the first lumen 36 to the exterior surface 32 of the outer wall 30 and provides fluid communication between the first lumen 36 and the external environment at the exterior surface of the outer wall. Although four first apertures 48 are shown in FIG. 1, there may be more first apertures or fewer first apertures, including, for example, only one first aperture. The first apertures 48 may extend radially outward through the outer wall 30 along the shortest path between the exterior surface 32 and the interior surface 34. The shortest path may thus be oriented at 90° to a plane that is tangent to either the interior surface 34 or the exterior surface 32. Alternatively, the first apertures 48 may extend at an angle other than 90° to a plane that is tangent to either the interior surface 34 or the exterior surface 32 and may thus extend along a longer path between the exterior surface 32 and the interior surface 34. The consequence of such a longer path may be a first aperture 48 that is directed proximally or distally of the outer wall 30.

A series of second ports or second apertures 50 also extend through the outer wall 30 from the exterior surface 32 to the interior surface 34. The second apertures 50 are all located proximal of all of the first apertures 48 and proximal of the wall 46, which closes the second lumen 44. Each of the second apertures 50 extends from the second lumen 44 to the exterior surface 32 of the outer wall 30 and provides fluid communication between the second lumen 44 and the external environment at the exterior surface of the outer wall. Although four second apertures 50 are shown in FIG. 1, there may be more second apertures or fewer second apertures, including, for example, only one second aperture. The second apertures 50 may extend radially outward through the outer wall 30 along the shortest path between the exterior surface 32 and the interior surface 34. The shortest path may thus be oriented at 90° to a plane that is tangent to either the interior surface 34 or the exterior surface 32. Alternatively, the second apertures 50 may extend at an angle other than 90° to a plane that is tangent to either the interior surface 34 or the exterior surface 32 and may thus extend along a longer path between the exterior surface 32 and the interior surface 34. The consequence of such a longer path may be a second aperture 50 that is directed proximally or distally of the outer wall 30 and the cannula 16.

As previously indicated, an atraumatic member 20 may be mounted adjacent the distal end portion 22 and the distal end 26 of the cannula 16. The atraumatic member 20 may, for example, be a rounded tip (not shown), which is formed of a low durometer elastomer and which is secured to the distal end 26 of the cannula. Alternatively, as shown in FIG. 1, the atraumatic member 20 may be an inflatable balloon 52 formed of flexible material mounted on a hollow, tubular shaft 54, which is also formed of flexible material. The interior of the hollow shaft 54 provides a passageway for delivering fluid, such as a gas, to the interior of the balloon 52 to inflate the balloon. The interior of the hollow shaft 54 may, for example, communicate with the first lumen 36 of the cannula 16. With such a construction, a wall of flexible material defining the balloon 52 may also function as a wall closing the distal end of the first lumen 36. Regardless of the structure of the atraumatic member 20, however, the atraumatic member functions to help prevent injury to a patient when the cannula 16 is inserted into the patient, as will be described in greater detail below.

In order to be used to de-air a patient's heart, the cannula 16 must be connected in fluid communication with the source of fluid 12 and the source of suction 14. While there are numerous mechanisms and techniques for providing fluid communication between the cannula 16 and both the source of fluid 12 and the source of suction 14, the system 10 of FIG. 1 uses the control apparatus 18 to provide the fluid communication. The control apparatus 18 includes a housing 56. The housing 56 is elongated and includes rounded external surfaces to permit the housing to be grasped comfortably by a user of the system 10 and thus provide a handle. At a distal end 58, the housing 56 is attached to the proximal end 28 of the cannula 16. The attachment may be a butt attachment or the proximal end 28 and a part of the proximal end portion 24 of the cannula 16 may be received in the housing 56. At an opposite proximal end 60 of the housing 56, a first connecting tube 62 and a second connecting tube 64 project from the housing. Fittings 66 and 68 on the projecting ends of the first and second connecting tubes 62 and 64, respectively, permit the source of fluid 12 and the source of suction 14 to be connected in fluid communication with the first and second connecting tubes 62 and 64, respectively.

Within the housing 56 of the control apparatus 18 are fluid passageways (not shown) and valves (not shown) for placing the first and second connecting tubes 62 and 64 and thus the source of fluid 12 and the source of suction 14 in fluid communication with one or both of the first and second lumens 36 and 44 of the cannula 16. The valves (not shown) may be operated by manually engageable members 70 and 72, which may, for example, be rocker switches or sliding switches. The control apparatus 18 is configured to be operable to place the source of fluid 12, which fluid may be a gas, such as carbon dioxide, in fluid communication with one or both or neither of the first lumen 36 and the second lumen 44 of the cannula 16. The control apparatus 18 is also configured to be operable to place the source of suction 14 in fluid communication with one or both or neither of the first lumen 36 and the second lumen 44 of the cannula 16. The first lumen 36 may thus be placed in fluid communication with either or neither of the source of fluid 12 or the source of suction 14 independently of whether the second lumen 44 is placed in fluid communication with either or neither of the source of fluid or the source of suction. To achieve such operation, the control apparatus 18 may, for example, include a pair of four-position valves (not shown) connected via fluid passageways (not shown) within the housing 56 to the connecting tubes 62 and 64 and to the first and second lumens 36 and 44 of the cannula 16. Any valves (not shown) in the control apparatus 18 may be manually operable valves or electrically operable valves, such as solenoid valves.

Although the control apparatus 18 may be configured so as to provide just manually initiated fluid flow control between the cannula 16 and the sources of fluid and suction 12 and 14, respectively, the control apparatus may be configured to provide more automated fluid flow control. For example, the control apparatus 18 may incorporate a controller 78 that is electrically or wirelessly connected to sensors (not shown) and that is operably and electrically connected to electrically operated valves (not shown). Such a controller 78 could operate the valves based on information from the sensors. The sensors may, for example, be sensors for measuring pressure and/or carbon dioxide levels within either of the first and second lumens 36 and 44 when connected to the source of suction 14. The sensors may alternatively or additionally be sensors for measuring fluid flow rate, oxygen levels, nitrogen levels, and/or pH within either of the first and second lumens 36 and 44. The control apparatus 18 may also include a wired or wireless connection to receive data from an external device, such as an arterial blood gas analyzer. Further, the controller 78 may be pre-programmed or programmable to cycle the fluid control valves (not shown) through a particular routine of open and closing the valves over specified time intervals or until a particular sensor reading is achieved.

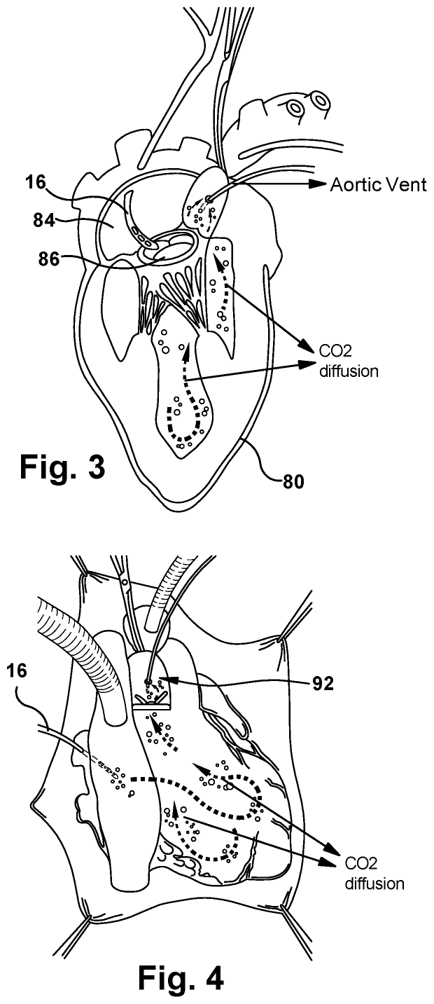

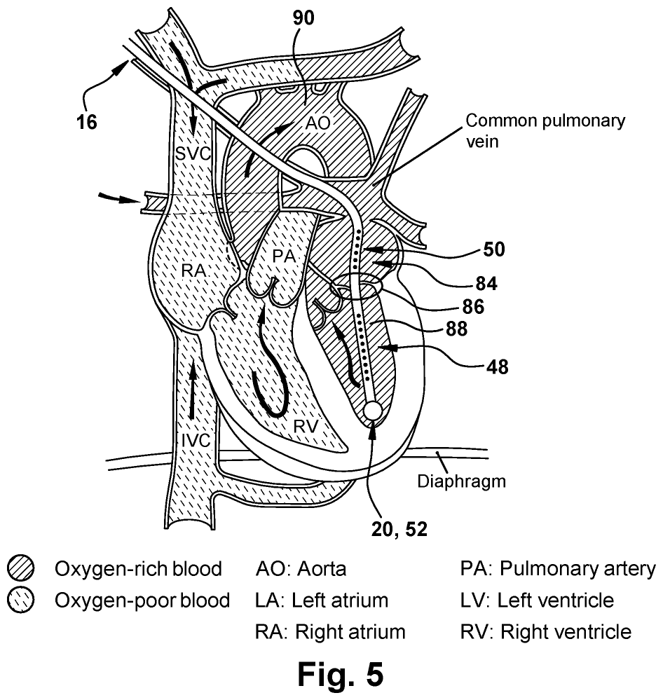

To use the system 10, the control apparatus 18 is connected to the source of fluid 12 and to the source of suction 14 by connecting lengths of tubing or other conduits 74 and 76 that extend from the source of fluid and the source of suction to the fitting 66 and the fitting 68 on the projecting ends of the first and second connecting tubes 62 and 64, respectively, of the control apparatus. The distal end 26 of the cannula 16, together with the atraumatic member 20, if used, is then inserted by the surgeon or other healthcare provider through a surgical incision into either the superior or the inferior right pulmonary vein and advanced to the mitral valve. As shown in FIGS. 3 and 4, the cannula 16 has been inserted into the superior right pulmonary vein of a patient's heart 80 and is being advanced through the left atrium 84 toward the mitral valve 86. As shown in FIG. 5, the cannula 16 has been inserted through the mitral valve 86 into the left ventricle 88.

During surgery, such as a cardiopulmonary bypass procedure, both the first apertures 48 and the second apertures 50 are connected to the source of suction 14 so as to cause the cannula 16 to vent and/or drain the left atrium 84 and the left ventricle 88. After the cardiopulmonary bypass procedure, but prior to releasing a clamp placed on the aorta 90, the patient (not shown) is placed in the Trendelenburg position with the patient's head disposed below the patient's feet. A separate aortic vent 92, which was previously inserted into the aorta 90, is then connected to a source of continuous suction (not shown). Fluid, specifically, carbon dioxide gas, from the source of fluid 12 is delivered through the second lumen 44 of the cannula 16 and the second apertures 50 and introduced into the left atrium 84. As the patient is in the Trendelenburg position, carbon dioxide gas introduced into the left atrium 84 will migrate through the mitral valve 86 into the left ventricle 88. In the left ventricle 88, the first lumen 36 and thus the first apertures 48 are connected to the source of suction 14 so that carbon dioxide gas that migrates to the left ventricle will be vented. After about 30 to about 60 seconds, the connections may be reversed so that carbon dioxide gas is introduced into the left ventricle 88 and is vented from the left atrium 84.

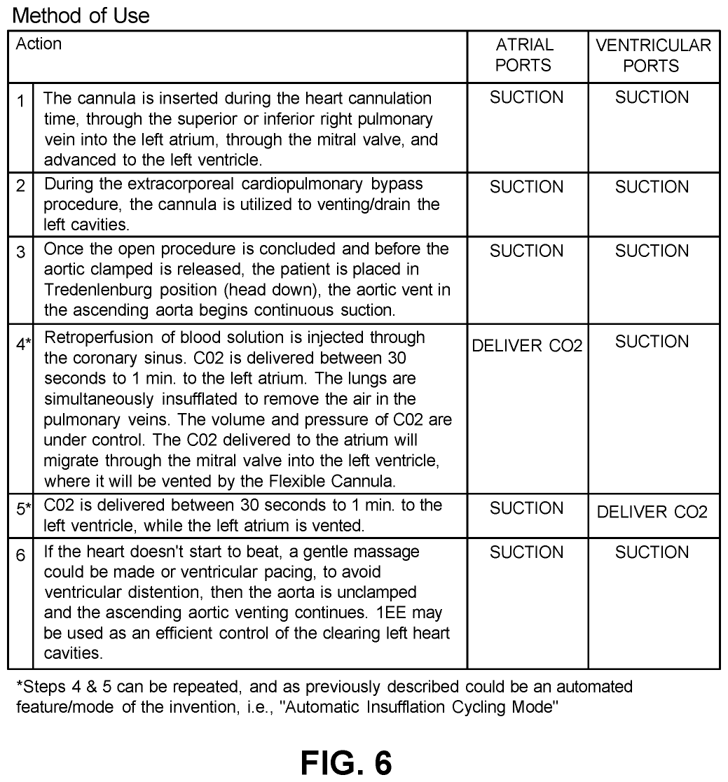

Although one specific method of using the system 10 is described in the preceding paragraphs, other methods of using the system are possible. For example, FIG. 6 presents steps in a somewhat modified or more detailed method of using the system 10.

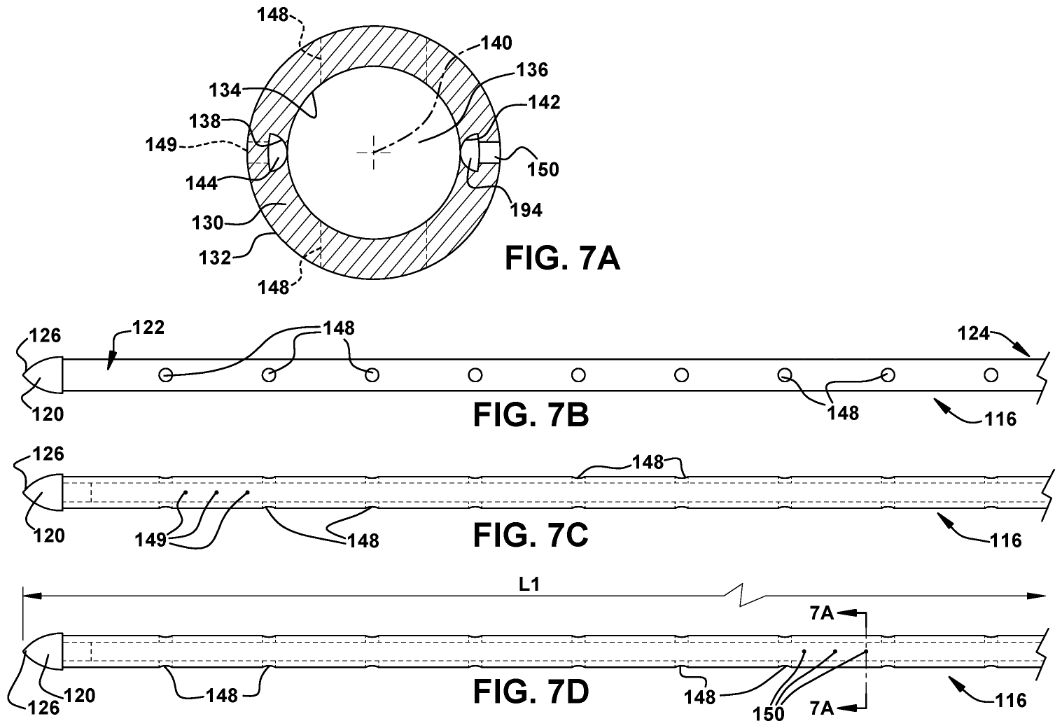

As illustrated in FIGS. 1-6 and as described above, the cannula 16 of an aspect of the present invention may have only two lumens, namely, first lumen 36 and second lumen 44. The cannula 16 may, however, have more than two lumens, as will be described below with reference to FIGS. 7A-9. For example, the cannula 16 may include a third lumen substantially similar to the second lumen 44 in configuration and dimensions, but located in any desired position; e.g., diametrically opposite the second lumen 44. Such a third lumen would, for example, permit carbon dioxide gas to be introduced into the left atrium 84 of a patient's heart 80 in two directions, thereby potentially increasing the delivery of carbon dioxide gas to and distribution of carbon dioxide gas in the patient's heart 80. Including more than two lumens 36, 44 in the catheter 16 may require the control apparatus 18 to have more valves and more manually engageable members 70 and 72. One of ordinary skill in the art can readily provide a suitable cannula 16 having any desired number of lumens, according to the teachings of the present application, for a particular use embodiment.

FIGS. 7A-9 illustrate a second embodiment of a cannula 116 suitable for use in a system such as the system 10 of FIG. 1. The cannula 116 of FIGS. 7A-9 is similar to the cannula 16 of FIGS. 1-6 and therefore, structures of FIGS. 7A-9 that are the same as or similar to those described with reference to FIGS. 1-6 have the same reference numbers, or the same reference numbers incremented by 100. Description of common elements and operation similar to those in the previously described first embodiment will not be repeated with respect to the second embodiment, but should instead be considered to be incorporated below by reference as appropriate.

In FIGS. 7A-9, the cannula 116 has a distal end portion 122 and a proximal end portion 124, which is spaced apart from the distal end portion. The cannula 116 also has a length L1, which extends from the distal end portion 122 to the proximal end portion 124. More particularly, the length L1 of the cannula 116 extends from the distal end 126 of the cannula to the proximal end (not shown) of the cannula.

As seen in FIG. 7A, the cannula 116 includes a tubular wall 130, which extends along the length L1 of the cannula. The wall 130 is made of a flexible biocompatible material, for example, a biocompatible plastic, such as silicone. The wall 130 has an exterior surface 132, which circumscribes the wall, and a first interior surface 134, which is coaxial with the exterior surface. The exterior surface 132, when viewed in cross-section taken perpendicular to the length L1 of the cannula 116, may be circular in shape, as shown in FIG. 7A, and the first interior surface 134, when viewed in cross-section taken perpendicular to the length L1 of the cannula 116, may also be circular in shape, as also shown in FIG. 7A. The exterior surface 132 and the first interior surface 134 may have other closed configurations or shapes, when viewed in cross-section taken perpendicular to the length L1 of the cannula 116, as desired.

The first interior surface 134 of the wall 130 of the cannula 116 defines a first lumen 136 of the cannula. The first lumen 136 extends along the length L1 of the cannula 116 and the length of the wall 130 from the proximal end portion 124 into the distal end portion 122. The first lumen 136 also extends along the length of the cannula 116 from the proximal end (not shown) to the distal end 126. The distal end of the first lumen 136 is closed. The distal end of the first lumen 136 may, for example, be closed by a wall or surface of an atraumatic plug or member 120, which extends transversely of the first lumen and the wall 130 adjacent the distal end 126 and the distal end portion 122 of the cannula 116.

The wall 130 also has a second interior surface 138. The second interior surface 138 extends parallel to the exterior surface 132 and the first interior surface 134, but is radially offset from a central longitudinal axis 140 of the wall 130 and the exterior surface 132. The second interior surface 138, when viewed in cross-section taken perpendicular to the length L1 of the cannula 116, may have any suitable shape, including, but not limited to, the “D” shaped cross-section shown in FIG. 7A. The second interior surface 138 of the wall 130 of the cannula 116 defines a second lumen 144 of the cannula. The second lumen 144 extends along the length L1 of the cannula 116 and the length of the wall 130 from the proximal end portion 124 into the distal end portion 122. The second lumen 144 also extends along the length of the cannula 116 from the proximal end (not shown) to the distal end 126. The distal end of the second lumen 144 is closed. The distal end of the second lumen 144 may, for example, be closed by a wall or surface of the atraumatic plug or member 120, which extends transversely of the second lumen and the wall 130 adjacent the distal end 126 and the distal end portion 122 of the cannula 116.

The wall 130 further has a third interior surface 142. The third interior surface 142 thus extends parallel to the exterior surface 132 and the first interior surface 134, but is radially offset from the central longitudinal axis 140 of the wall 130 and the exterior surface 132. The third interior surface 142, when viewed in cross-section taken perpendicular to the length L1 of the cannula 116, may have any suitable shape, including, but not limited to, the “D” shaped cross-section shown in FIG. 7A. The third interior surface 142 of the wall 130 of the cannula 116 defines a third lumen 192 of the cannula. The third lumen 192 extends along the length L1 of the cannula 116 and the length of the wall 130 from the proximal end portion 124 into the distal end portion 122. The third lumen 192 also extends along the length of the cannula 116 from the proximal end (not shown) to the distal end 126. The distal end of the third lumen 192 is closed. The distal end of the third lumen 192 may, for example, be closed by a wall or surface of the atraumatic plug or member 120, which extends transversely of the second lumen and the wall 130 adjacent the distal end 126 and the distal end portion 122 of the cannula 116.

In the distal end portion 122 of the cannula 116, a series of first ports or first apertures 148 extend through the wall 130 from the exterior surface 132 to the first interior surface 134. Each of the first apertures 148 thus extends from the first lumen 136 to the exterior surface 132 of the wall 130 and provides fluid communication between the first lumen 136 and the external environment at the exterior surface of the outer wall. There may be any desired number of first apertures 148, including, for example, only one first aperture. As shown in FIG. 7A, the first apertures 148 are arranged in pairs with one first aperture of a pair of first apertures extending through one of two diametrically opposed portions of the wall 130 and the other first aperture of a pair of first apertures extending through the other of two diametrically opposed portions of the wall 130. Thus, for example, for each first aperture 148 shown in FIG. 7B, a second first aperture is disposed in the wall 130 opposite the first aperture visible in FIG. 7B.

The first apertures 148 may extend radially outward through the wall 130 along the shortest path between the exterior surface 132 and the first interior surface 134, as is shown in FIG. 7A. The shortest path may thus be oriented at 90° to a plane that is tangent to either the exterior surface 132 or the first interior surface 134. Alternatively, the first apertures 148 may extend at an angle other than 90° to a plane that is tangent to either the exterior surface 132 or the first interior surface 134 and may thus extend along a longer path between the exterior surface 132 and the first interior surface 134. The consequence of such a longer path may be a first aperture 148 that is directed proximally or distally of the wall 130.

A series of second ports or second apertures 149 extend through the wall 130 from the exterior surface 132 to the second interior surface 138. The second apertures 149 are located in the distal end portion 122 of the cannula 116 and may all be located proximal of at least some of the first apertures 148. Each of the second apertures 149 extends from the second lumen 144 to the exterior surface 132 of the wall 130 and provides fluid communication between the second lumen 144 and the external environment at the exterior surface of the wall. Although three second apertures 149 are shown in FIG. 7C, there may be more second apertures or fewer second apertures, including, for example, only one second aperture. The second apertures 149 may extend radially outward through the wall 130 along the shortest path between the exterior surface 132 and the second interior surface 138. The shortest path may thus be oriented at 90° to a plane that is tangent to either the exterior surface 132 or the second interior surface 138. Alternatively, the second apertures 149 may extend at an angle other than 90° to a plane that is tangent to either the exterior surface 132 or the second interior surface 138 and may thus extend along a longer path between the exterior surface 132 and the second interior surface 138. The consequence of such a longer path may be a second aperture 149 that is directed proximally or distally of the wall 130 and the cannula 116.

A series of third ports or third apertures 150 extend through the wall 130 from the exterior surface 132 to the third interior surface 142. The third apertures 150 are all located proximal of all of the second apertures 149 and proximal of at least some of the first apertures 148. Each of the third apertures 150 extends from the third lumen 192 to the exterior surface 132 of the wall 130 and provides fluid communication between the third lumen 192 and the external environment at the exterior surface of the outer wall. Although three third apertures 150 are shown in FIG. 7D, there may be more third apertures or fewer third apertures, including, for example, only one third aperture. The third apertures 150 may extend radially outward through the wall 130 along the shortest path between the exterior surface 132 and the third interior surface 142. The shortest path may thus be oriented at 90° to a plane that is tangent to either the exterior surface 132 or the third interior surface 142. Alternatively, the third apertures 150 may extend at an angle other than 90° to a plane that is tangent to either the exterior surface 132 or the third interior surface 142 and may thus extend along a longer path between the exterior surface 132 and the third interior surface 142. The consequence of such a longer path may be a third aperture 150 that is directed proximally or distally of the wall 130 and the cannula 116.

As previously indicated, the atraumatic member 120 may be mounted adjacent the distal end portion 122 and the distal end 126 of the cannula 116. The atraumatic member 120 may, for example, be a rounded tip, which is formed of a low durometer elastomer and which is secured to the distal end 126 of the cannula 116. Alternatively, the atraumatic member 120 may be an inflatable balloon (not shown) formed of flexible material mounted on a hollow, tubular shaft (not shown), which is also formed of flexible material. The interior of the hollow shaft provides a passageway for delivering fluid, such as a gas, to the interior of the balloon to inflate the balloon. The interior of the hollow shaft may, for example, communicate with the first lumen 136 of the cannula 116. With such a construction, a wall of flexible material defining the balloon may also function as a wall closing the distal end of the first lumen 136. Regardless of the structure of the atraumatic member 120, however, the atraumatic member functions to help prevent injury to a patient when the cannula 116 is inserted into the patient, as described in greater detail previously.

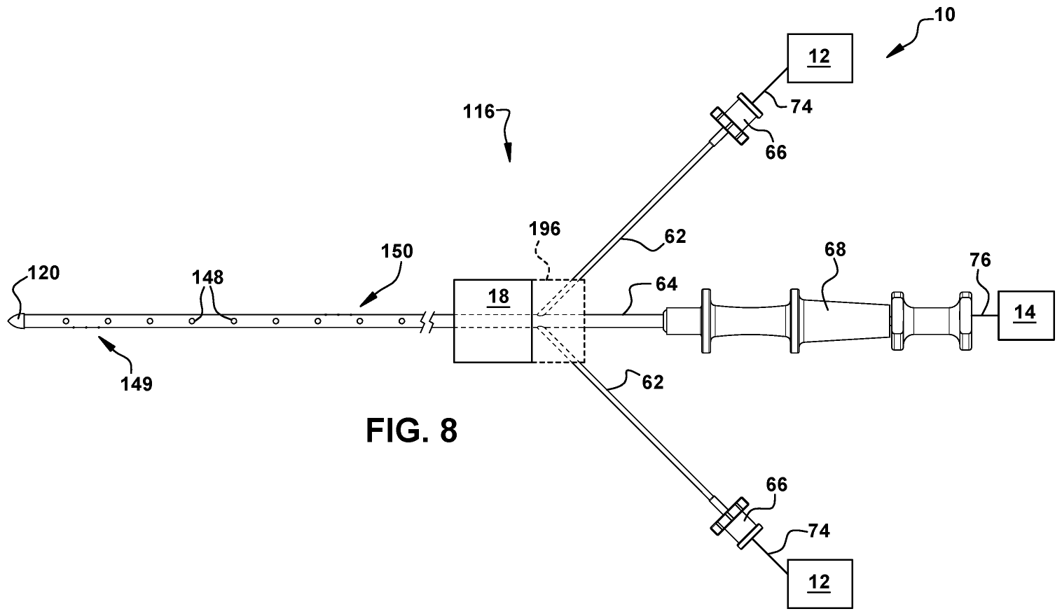

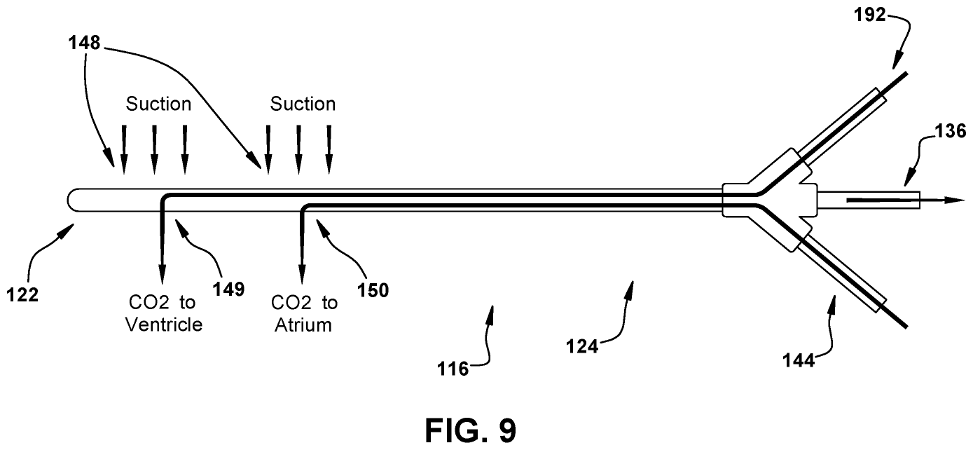

In use, as shown schematically in FIGS. 8-9, the first apertures 148 of the cannula 116 may be connected to a source of suction 14. The second apertures 149 and the third apertures 150 may each be connected to at least one source of fluid 12, such as carbon dioxide gas. Fluid from at least one source of fluid 12 may be delivered through the second lumen 144 of the cannula 116 and the second apertures 149 and introduced into the left ventricle of a patient. Fluid from the source of fluid 12 may be delivered through the third lumen 192 of the cannula 116 and the third apertures 150 and introduced into the left atrium of a patient. At one or more times during a surgical procedure, however, the second apertures 149 and/or the third apertures 150 may be connected to a source of suction 14 for removing fluid from an adjacent area of a patient's heart 80.

Although the second lumen 144 and the third lumen 192 of the cannula 116 are described as extending along the length of the cannula 116 from the proximal end (not shown) to the distal end 126 and as being closed by the atraumatic tip 120, either or both of the second lumen and the third lumen may extend for a shorter distance and be closed, for example, by a transverse wall, like the second lumen 44 of the cannula 16. With reference to FIG. 8, a schematic view of a system 10 including the cannula 116 of the second embodiment is shown. As indicated by a dashed line, a control apparatus 18 may be present; otherwise, the first, second, and third lumens 136, 144, and 192 may be attached directly, or via intervening couplers or fittings 66, 68 to at least one source of fluid 12 and/or at least one source of suction 14, as desired for a particular use environment of the system 10 including the cannula 116. Any desired number (if any) and configuration(s) of first and second connecting tubes 62, 64 and/or tubing 74, 76 may be provided to facilitate desired operation of the system 10. For example, it is contemplated that the first lumen 136 could be connected to a source of suction 14 and the second and third lumens 144 and 192 could be connected to a single, or individual, source(s) of fluid 12, as desired. It is also contemplated that a manifold (shown schematically at 196) of any desired configuration could selectively connect any or all of the first, second, and third lumens 136 to at least one of the source of fluid 12 and the source of suction 14, concurrently or sequentially. The manifold 196, when present, may be user-manipulable manually or automatically to provide desired fluid and/or suction functions to any one or more of the first, second, and third apertures 148, 149, 150 in any desired sequence during operation of the system 10 in de-airing a patient's heart 80 for a particular use environment.

FIG. 9 schematically depicts provision of carbon dioxide to the left atrium 84 and left ventricle 88 of the patient's heart 80 while suction is being applied at multiple locations along the length of the cannula 116 to remove unwanted air.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

We claim:

1. A cannula for de-airing a patient's heart following surgery, the cannula comprising
a tubular wall having a distal end portion and a proximal end portion, the wall also having a length extending from the distal end portion to the proximal end portion, the wall at least partially defining a first lumen extending lengthwise of the wall into the distal end portion and terminating in a closed first end, the wall also at least partially defining a second lumen extending lengthwise of the wall and terminating in a closed second end, the wall being configured and dimensioned such that the cannula can be inserted into the patient's heart;
a first aperture extending through the wall from the first lumen to an exterior surface of the wall; and
a second aperture extending through the wall from the second lumen to the exterior surface of the wall.

2. The cannula of claim 1, wherein the second end is located proximally of the closed first end of the first lumen.

3. The cannula of claim 1, wherein the second aperture extends through the distal end portion of the wall.

4. The cannula of claim 1, wherein the wall at least partially defines a third lumen extending lengthwise of the wall and terminating in a closed third end, the cannula including a third aperture extending through the wall from the third lumen to the exterior surface of the wall.

5. The cannula of claim 1, wherein the second aperture is located proximal of the first aperture.

6. The cannula of claim 3, wherein the second aperture is located proximal of the first aperture and of the third aperture.

7. The cannula of claim 1, including a manifold selectively connecting each of the first and second lumens to at least one of a source of fluid and a source of suction for de-airing the patient's heart.

8. The cannula of claim 7, wherein the fluid is carbon dioxide.

9. The cannula of claim 1, including a first fitting connecting the first lumen to a source of suction and a second fitting connecting the second lumen to a source of fluid.

10. The cannula of claim 3, including a first fitting selectively connecting the first lumen to a source of suction, a second fitting selectively connecting the second lumen to a source of fluid, and a third fitting selectively connecting the third lumen to the source of fluid.

11. The cannula of claim 1, including an atraumatic member distally adjacent to the closed first end.

12. A system for de-airing a patient's heart following surgery comprising:
a source of fluid;
a source of suction;
a cannula configured and dimensioned to be selectively inserted into the patient's heart and to selectively conduct fluid from the source of fluid to the patient's heart, the cannula having a distal end portion and a proximal end portion, the cannula also having a length extending from the distal end portion to the proximal end portion, the cannula including a wall extending lengthwise of the cannula, the wall at least partially defining a first lumen extending lengthwise of the cannula into the distal end portion and terminating in a closed first end, the wall also at least partially defining a second lumen extending lengthwise of the cannula and terminating in a closed second end located proximally of the closed first end of the first lumen, the cannula also including a first aperture extending through the wall of the cannula from the first lumen to an exterior surface of the wall and a second aperture extending through the wall of the cannula from the second lumen to the exterior surface of the wall; and
a control apparatus operable to control pressure and flow of fluid from the source of fluid to the cannula and to control application of suction from the source of suction to the cannula, the control apparatus being in fluid communication with the source of fluid and with the cannula, the control apparatus also being in fluid communication with the source of suction and with the cannula, the control apparatus being operable to direct fluid from the source of fluid to the first lumen and/or the second lumen of the cannula, the control apparatus also being operable to apply suction from the source of suction to the first lumen and/or the second lumen.

13. The system of claim 12, including an atraumatic member mounted on a distal end of the cannula.

14. The system of claim 12, wherein the control apparatus includes manually engagable members connected to the proximal end portion of the cannula to at least partially control operation of the control apparatus.

15. The system of claim 12, including a manifold selectively connecting each of the first and second lumens to at least one of a source of fluid and a source of suction for de-airing the patient's heart.

16. The system of claim 12, wherein the wall at least partially defines a third lumen extending lengthwise of the wall and terminating in a closed third end, the cannula including a third aperture extending through the wall from the third lumen to the exterior surface of the wall.

17. The system of claim 16, including a manifold selectively connecting each of the first, second, and third lumens to at least one of a source of fluid and a source of suction for de-airing the patient's heart.

18. The system of claim 12, including a first fitting connecting the first lumen to the source of suction and a second fitting connecting the second lumen to the source of fluid.

19. The system of claim 16, including a first fitting selectively connecting the first lumen to the source of suction, a second fitting selectively connecting the second lumen to the source of fluid, and a third fitting selectively connecting the third lumen to the source of fluid.

20. A method of de-airing a patient's heart following surgery, the method comprising the steps of:
placing a patient in a Trendelenburg position with the patient's head disposed below the patient's feet;
inserting a plural lumen cannula into a left atrium of the patient's heart, the plural lumen cannula comprising a tubular wall at least partially defining a first lumen and at least partially defining a second lumen, a first aperture being formed in the wall adjacent a distal end of the plural lumen cannula and communicating with the first lumen, a second aperture being formed in the wall proximal of the first aperture and communicating with the second lumen;
extending the plural lumen cannula into a left ventricle of the patient's heart such that the first aperture is disposed in the left ventricle and the second aperture is located in the left atrium;
introducing fluid into the left atrium through the second lumen and the second aperture; and
suctioning fluid from the left ventricle through the first aperture and the first lumen.

21. The method of claim 20, wherein the tubular wall of the plural lumen cannula at least partially defines a third lumen, a third aperture being formed in the wall distal of the second aperture and communicating with the third lumen, the method comprising the step of introducing fluid into the left ventricle through the third lumen and the third aperture.
